(19) United States
(12) Patent Application Publication
(10) Pub. No.: US 2009/0311189 A1
(43) Pub. Date: Dec. 17, 2009

(54) TEMPERATURE-JUMP DYNAMIC NUCLEAR POLARIZATION

(75) Inventors: Robert G. Griffin, Newton, MA (US); Kan-Nian Hu, Cambridge, MA (US); Chan Gyu Joo, Belmont, MA (US)

Correspondence Address: CHOATE, HALL & STEWART LLP, TWO INTERNATIONAL PLACE, BOSTON, MA, 02110, US

(73) Assignee: Massachusetts Institute of Technology, Cambridge, MA (US)

(21) Appl. No.: 12/300,524

(22) PCT Filed: May 10, 2007
(86) PCT No.: PCT/US07/68654

§ 371 Date: Apr. 15, 2009

Related U.S. Application Data
(60) Provisional application No. 60/747,098, filed on May 12, 2006.

(51) Int. Cl. A61K 49/06; G01R 33/48

(52) U.S. Cl. 424/9.3; 324/309

(57) ABSTRACT

In one aspect, the present invention provides a method for enhancing the sensitivity of liquid-state NMR or MRI experiments. In general, the method involves providing a frozen sample in a magnetic field, wherein the frozen sample includes a polarizing agent with at least one unpaired electron and an analyte with at least one spin half nucleus; polarizing the at least one spin half nucleus of the analyte by irradiating the frozen sample with radiation having a frequency that excites electron spin transitions in the at least one unpaired electron of the polarizing agent; melting the frozen sample to produce a molten sample; and (d) detecting nuclear spin transitions in the at least one spin half nucleus of the analyte in the molten sample. In certain embodiments, the methods further comprise a step of freezing a sample in a magnetic field to provide the frozen sample in a magnetic field.

### PRIORITY CLAIM

This application claims priority to U.S. Provisional Application Ser. No. 60/747,098 filed May 12, 2006, the contents of which are incorporated herein by reference.

### GOVERNMENT FUNDING

The inventions described herein were made with support from funding from the National Institutes of Health, Grant No. EB-002804. The U.S. Government therefore has certain rights in these invention.

### BACKGROUND OF THE INVENTION

The last decade has witnessed a renaissance in the development of approaches to prepare samples with high nuclear spin polarizations with the goal of increasing signal intensities in NMR spectra of solids and liquids. These approaches have included high frequency, microwave driven dynamic nuclear polarization (DNP)¹⁻⁹, para hydrogen induced polarization (PHIP)^{10,11}, polarization of noble gases such as He, Xe¹²⁻¹⁴ and more recently Kr¹⁵, and optically pumped nuclear polarization of semiconductors¹⁶⁻¹⁸ and photosynthetic reaction centers and other proteins¹⁹⁻²². Dynamic nuclear polarization is an approach in which the large spin polarization in an electron spin system is transferred to a nuclear spin reservoir via microwave irradiation of the electron paramagnetic resonance (EPR) spectrum. All of these approaches successfully yield highly polarized spins, and are studied to elucidate features of the polarization methods or of the material being polarized. However, one of the most appealing aspects of high polarization methods is the possibility of transferring the polarization from the source to a surrounding medium such as a solvent and to subsequently distribute the polarization to chemically, physically or biologically interesting solutes. For this to occur it is necessary that the polarizing agent be strongly coupled to the lattice of nuclear spins, and in this regard paramagnetic polarizing agents are appealing since the large magnetic moment of the electron spin couples effectively to its surrounding nuclei. Accordingly, high frequency microwave (≧100 GHz) driven DNP experiments using stable free radicals as polarizing agents^{2, 3} are currently used successfully to polarize a variety of systems including solid polymers^{4, 23-27}, frozen solutions of small molecules³, amino acides^{5,6}, virus particles⁷, soluble and membrane proteins⁸ and amyloid nanocrystals⁹ achieving enhancements in the range of 50-400, depending on the details of the experiment. Superscript numbers refer to the attached reference list. The contents of all of these references are incorporated herein by reference.

In addition to polarizing solid samples, there is considerable interest in using high frequency DNP to enhance the sensitivity of liquid state nuclear magnetic resonance (NMR) experiments. However, the polarization mechanisms operative in dielectric solids at high fields—the solid effect^{28, 29}, the cross effect^{2, 30} and thermal mixing²⁸—are not applicable to liquids. Instead, the Overhauser effect (OE)^{31, 32} is the dominant polarization mechanism, and it is efficient only at low magnetic fields. In particular, for small molecules, the rotational or translational correlation times are˜10⁻¹²s and at low magnetic fields the condition ωₛτ_{c}≦1 is satisfied (where ωₛ, is the electron Larmor frequency and τ_{c} the correlation time), and the Overhauser effect is effective in transferring polarization. However, in the high field regime commonly employed in contemporary NMR experiments, ωₛ is large, the rotational and translational spectral densities are vanishingly small, and the Overhauser enhancements decrease significantly³³. Thus, to enhance the polarization of liquid samples in high field experiments, an alternative method is required.

### SUMMARY OF THE INVENTION

In one aspect, the present invention provides a method for enhancing the sensitivity of liquid-state NMR or MRI experiments. In general, the method involves (a) providing a frozen sample in a magnetic field, wherein the frozen sample includes a polarizing agent with at least one unpaired electron and an analyte with at least one spin half nucleus; (b) polarizing the at least one spin half nucleus of the analyte by irradiating the frozen sample with radiation having a frequency that excites electron spin transitions in the at least one unpaired electron of the polarizing agent; (c) melting the frozen sample to produce a molten sample; and (d) detecting nuclear spin transitions in the at least one spin half nucleus of the analyte in the molten sample. In certain embodiments, the methods further comprise a step of freezing a sample in a magnetic field to provide the frozen sample in a magnetic field. In one such embodiment, the freezing, polarizing, melting and detecting steps are repeated at least once.

In general, the methods may be used to polarize any analyte. Without limitation, the analyte may be a protein or nucleic acid. Numerous liquid-state NMR methods have been developed to study the structures of these biomolecules, e.g., one dimensional techniques, multi-dimensional techniques, including without limitation techniques that rely on NOESY, ROESY, TOCSY, HSQC, HMQC, etc. type polarization transfers and combinations thereof. Any of these techniques and variants thereof may benefit from the enhanced NMR signals that can be provided by the inventive methods. The inventive methods may also be advantageously used to improve the detection of analytes (e.g., metabolites) that are present in a sample at low concentrations. Currently, the lower limit of detection by convention liquid-state NMR is on the order of about 10 μM. Since the DNP enhancement provided by the present invention may range from 2 to 10,000 or more (depending on the temperature, magnetic field, etc.) the inventive methods enable the detection of less than 1 μM, less than 100 nM, less than 10 nM or even less than 1 nM of a metabolite or other analyte of interest. When the analyte is being used as an imaging agent for an MRI experiment then it will preferably include at least one spin half nucleus with a long T₁ relaxation time. This will ensure that the enhancement is not lost by relaxation in between the polarizing and detecting steps. For example, U.S. Pat. No. 6,311,086 (the contents of which are incorporated herein by reference) describes imaging agents that include spin half nuclei with T₁ relaxation times of at least 6 seconds at 310 K in D₂O in a magnetic field of 7 T. It will be appreciated that any of the imaging agents that are described in U.S. Pat. No. 6,311,086 may be used as an analyte in an inventive method. It is also to be understood that any known MRI technique may be used to image the spatial distribution of a polarized analyte once administered to a subject (e.g., see MRI in Practice Ed. by Westerbrook et al., Blackwell Publishing, Oxford, UK, 2005, the contents of which are incorporated herein by reference).

Any spin half nucleus within the analyte may be polarized according to the inventive methods. In one embodiment, the spin half nucleus is a ¹H nucleus. In one embodiment, the spin half nucleus is a ¹³C nucleus. In one embodiment, the spin half nucleus is a ¹⁵N nucleus. In one embodiment, the spin half nucleus is a ¹⁹F nucleus. The spin half nucleus may be present in the analyte at natural abundance levels. Alternatively, stronger signals may be obtained if the spin half nucleus (e.g., ¹³C, ¹⁵N, ¹⁹F, etc.) is enriched at one or more positions within the analyte. A variety of methods are known in the art for enriching one or more sites of an analyte (e.g., a protein, nucleic acid, metabolite, imaging agent, etc.). When the at least one spin half nucleus has a γ-value smaller than that of ¹H (e.g., ¹³C, ¹⁵N, ¹⁹F, etc.) then in certain embodiments, the step of polarizing may further involve irradiating the frozen sample with radiation having a frequency that causes cross-polarization between a ¹H nucleus present in the sample (e.g., without limitation from ¹H₂O) and the at least one spin half nucleus of the analyte.

The inventive methods may be performed under any magnetic field strength. In one embodiment the field may have a strength in the range of about 0. 1 T to about 30 T. For example, some of the experiments that are described herein were performed at 5 T. The radiation for exciting electron spin transitions in the unpaired electron(s) of the polarizing agent at these fields will be in the range of about 2.8 GHz to about 840 GHz. For examples, the radiation in the experiments that are described herein was from a 140 GHz gyrotron. In certain embodiments, the polarization step may take less than about 2 minutes, e.g., less than about 90 seconds or less than about 1 minute.

In certain embodiments, the sample may be recycled by freezing the sample, repolarizing the at least one spin half nucleus of the analyte by irradiating the frozen sample with radiation having a frequency that excites electron spin transitions in the biradical, remelting the frozen sample to produce a molten sample, and redetecting nuclear spin transitions in the at least one spin half nucleus of the analyte in the molten sample. This method can be repeated for as many cycles as needed. This can be used, e.g., to signal average NMR signals and thereby further enhance the sensitivity of a liquid-state NMR experiment. The freezing step can generally be achieved by cooling the sample until it reaches a solid state. In certain embodiments, the sample can be cooled to a temperature of less than about 200 K. For example, the sample may be cooled to a temperature in the range of about 1 K to about 100 K. Some of the experiments that are described herein involved cooling the sample to a temperature of about 90 K. In one embodiment, the freezing step may be completed in less than about 2 minutes, e.g., less than about 90 seconds, or less than about 1 minute.

In general, once a frozen sample has been polarized according to the present invention it can be melted using any suitable method. In certain embodiments, this is achieved by exposing the frozen sample to radiation having a wavelength of less than about 100 μm, e.g., in the range of about 0.5 μm and about 50 μm. In one embodiment, the radiation may come from a laser, e.g., a CO₂ laser. In another embodiment, the radiation may come from a lamp, e.g., an infra-red lamp. The frozen sample can be exposed to the radiation using an optical fiber. This will typically involve coupling the radiation (e.g., from a laser or lamp) to one end of the fiber, e.g., using a lens. In one embodiment, the sample is within a cylindrical rotor. Advantageously, the rotor can be made of quartz which allows both microwave radiation (e.g., the 140 GHz radiation from a gyrotron) and infra-red radiation (e.g., from a CO₂ laser) to reach the sample. We have also found that a quartz rotor does not crack when exposed to multiple freeze-thaw cycles. Finally, the use of a cylindrical rotor enables the sample to be spun during the melting step (and optionally during other steps including the detecting step) which we have found to significantly improve melting homogeneity and time. In the experiments that are described herein we were able to melt samples in less than about 1 second.

Once melted, the molten sample may be analyzed by liquid-state NMR. Any liquid-state NMR technique can be used to detect the polarized nucleus or nuclei, e.g., one dimensional techniques, multi-dimensional techniques, including without limitation techniques that rely on NOESY, ROESY, TOCSY, HSQC, HMQC, etc. type polarization transfers and combinations thereof. The detected NMR signals may be from any spin half nucleus of the analyte, e.g., ¹H, ¹³C, ¹⁵N etc. In certain embodiments it may prove advantageous to decouple the polarized nucleus or nuclei from ¹H nuclei present in the sample.

Alternatively, in certain embodiments, at least a portion of the molten sample may be administered to a subject and then imaged by MRI. According to this last embodiment, the administered portion of the molten sample includes an amount of the polarized analyte. In certain embodiments (e.g., when the biradical is toxic) the polarized analyte may be separated from the biradical prior to administration. U.S. Pat. No. 6,311,086 (the contents of which are incorporated herein by reference) describes several methods for achieving such a separation (e.g., physical and chemical separation or extraction techniques). The polarized analyte may be administered to a subject using any known route of administration (e.g., by injection, ingestion, inhalation, etc.).

In general, any polarizing agent with an unpaired electron may be used according to the inventive methods. In certain embodiments, the polarizing agent is a monoradical, e.g., any one of the nitrogen oxide radicals (e.g., TEMPO based radicals) and trityl radicals that have been described in the art. In other embodiments, the polarizing agent is a biradical as further described herein and in U.S. Patent Publication No. 20050107696, the entire contents of which are incorporated herein by reference. Without limitation, in one embodiment, the polarizing agent is bis-TEMPO-2-ethyleneglycol (BT2E). In another embodiment, the polarizing agent is a biradical described in a PCT patent application that we filed on May 10, 2007 entitled “Biradical Polarizing Agents for Dynamic Nuclear Polarization”, the contents of which are incorporated herein by reference. In one embodiment, the polarizing agent is 1-(TEMPO-4-oxy)-3-(TEMPO-4-amino)-propan-2-ol (TOTAPOL).

In another aspect, the present invention also provides systems for performing these methods. Thus in one aspect a system is provided that comprises an NMR spectrometer, an NMR magnet including a probe for coupling radiofrequency radiation with a sample (e.g., a 5 T magnet), a microwave gyrotron (e.g., a 140 GHz source), a source of infra-red radiation (e.g., a CO₂ laser) and a quartz rotor for holding a sample. The system may further comprise an optical waveguide for delivering the microwave radiation to the quartz rotor. The system may also comprise an optical fiber for delivering the infra-red radiation to the quartz rotor.

### BRIEF DESCRIPTION OF THE DRAWING

FIG. 1 is a perspective view of one embodiment of a system for carrying out a method of the present invention.

FIG. 2 is a block diagram illustrating one embodiment of a temperature jump-DNP cycle. As shown, the TJ-DNP cycle may consist of cooling, polarization, melting, and acquisition steps. The microwaves for the DNP process were supplied by a 140 GHz gyrotron, and the melting was accomplished with a 10.6 μm CO₂ laser. With the current configuration of the apparatus, the experiment can be recycled every 60-90 s.

FIG. 3 is a schematic illustration of one embodiment of a pulse sequence for observing sensitivity enhanced liquid state NMR signals using temperature jump-DNP. The samples are irradiated with 140 GHz microwaves at 90 K, polarizing the ¹H spins in the sample. Enhanced ¹H polarization is then transferred to ¹³C via cross polarization. During the laser heating, the ¹³C magnetization is stored along the z-axis of the rotating frame. The ¹³C spectrum is detected following a 90° pulse in the presence of WALTZ ¹H decoupling.

FIG. 4 are temperature jump-DNP NMR spectra of selected experimental samples: (a) ¹³C-urea in 50% ²H₆-DMSO and 50% water (80% ²H₂O/20% H₂O), (b) Na[1,2-¹³C₂,²H₃]-acetate in 60% ²H₈-glycerol and 40% water (80% ²H₂O/20% H₂O), and (c) [U-¹³C₆,²H₇]-glucose in H₂O. Samples contained 3-5 mM TOTAPOL biradical polarizing agent, corresponding to 6-10 mM electrons. As explained in the text, deuteration of the samples was employed in order to circumvent the ¹H mediated ¹³C relaxation in the viscous solution phase. The times required for polarization and melting of the sample are indicated next to each trace. The TJ-DNP spectra (the top traces in each figure) were recorded with a single scan, while the room-temperature spectra were recorded with (a) 256, (b) 128, and (c) 512 scans, respectively.

FIG. 5 shows sixteen spectra of the carbonyl resonance in [U-¹³C]-L-proline resulting from a series of TJ-DNP experiments employing the sequence DNP (40 s)—melting (1 s)—acquisition (100 ms)—refreezing (90 s) (left). The spectra illustrate that, following melting, the sample can be refrozen and repolarized and another spectrum recorded in order to perform signal averaging (right). The 16 spectra can be averaged to show improved signal-to-noise.

### DESCRIPTION OF CERTAIN PREFERRED EMBODIMENTS

The inventive methods have been used to generate enhancements in the range of 140-280 in NMR spectra of low-γ spins such as ¹³C and ¹⁵N. In these experiments, we polarized the ¹H spins in various samples at low temperatures (˜90 K) using biradical polarizing agents^{2, 34}. The polarization was transferred to low-γ spins with cross polarization, the sample was melted with an infrared laser pulse, and the enhanced signal observed in the presence of decoupling. If the polarization step were to be performed at a lower temperature (e.g., 10 K), then an even larger enhancement factor would be observed.

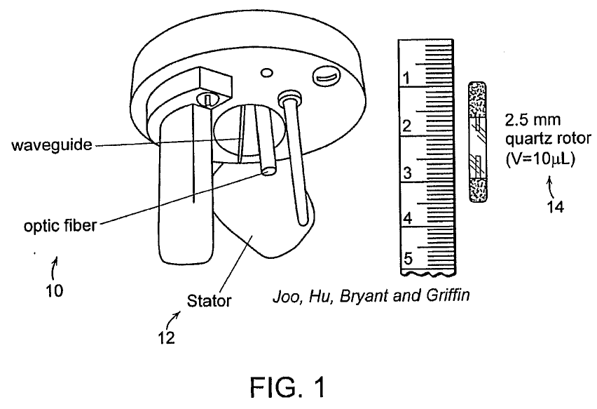

These experiments were carried out with the apparatus shown in FIG. 1. An optic fiber 10 delivered 10.6 μm radiation from a CO₂ laser (not shown) onto a sample. The sample was contained in a 2.5 mm quartz rotor 12 that can withstand temperature cycling that includes repeatedly cycling the sample between about 100 K where the dynamic nuclear polarization occurs and about 300 K where the liquid state NMR spectrum is observed. The figure shows a magic angle spinning stator 14. Experimental results will now be discussed.

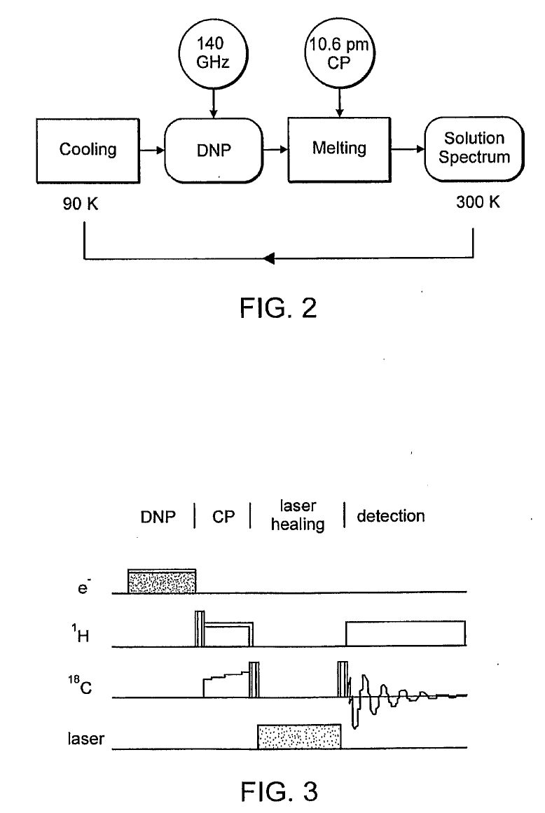

Samples for the experiments consisted of solutions containing high concentrations of ¹³C labeled small molecules to facilitate observation of signals in the absence of DNP. In particular, the high concentration facilitated observation of the signal intensity in the absence of microwave irradiation and therefore measurement of the enhancement. For example, in the experiments below we used 2 M ¹³C-urea in a solvent of composition 60% ²H₈-glycerol and 40% water (80% ²H₂O/20% H₂O). The solution was prepared with 3-5 mM TOTAPOL³⁴ as the biradical polarizing agent. About 9 μL of sample was placed in a 2.5 mm OD quartz capillary and NMR measurements were conducted in a custom designed probe in a 5 T magnet (211 MHz for ¹H and 53.31 MHz for ¹³C). Continuous microwave irradiation was generated with a 140 GHz gyrotron³⁵. The sample was maintained at 90 K by circulating cold N₂ gas during the experimental cycle. Typically the equilibrium polarization buildup required 15-30 s (the ¹H T₁ is typically 5-10 s), and the enhancement in the solid state spectra was 165 at this temperature and magnetic field. The rapid temperature jump (TJ) was performed by irradiating the sample with 10.6 μm radiation from the CO₂ laser transmitted to the sample through a multimode hollow optic fiber. Haw and coworkers^{36, 37} used a similar approach in TJ experiments on polymers with the exception that the sample was larger (5 mm diameter rotors) and required higher laser power. Thus, it was necessary to use lenses rather than an optic fiber to irradiate the sample. After melting, the solution NMR spectrum was recorded in the presence of decoupling, and the sample was refrozen and repolarized for another experimental cycle. Typically the freezing required 60 s, and the melting <1 s. FIG. 2 illustrates the cycle used in the TJ-DNP experiments—cooling, polarization with microwaves, melting with IR radiation, and observation of the liquid state NMR spectrum. FIG. 3 shows the pulse sequence associated with these steps and it incorporates storage/retrieval pulses prior to and following the melting step of the experiment.

Enhancements, ε^{†}, (vide infra) were determined by comparing the signal intensities of the DNP enhanced ¹³C signal intensities obtained in the melting experiment with those obtained from room temperature solution NMR experiments. The room temperature liquid state spectra were directly detected and typically acquired by averaging 1024 scans with a long recycle delay (60-120 s) to ensure that we reached the equilibrium Boltzmann polarization. Note that in generating the ¹³C DNP enhanced signals, we transferred polarization from electrons to ¹H and then to ¹³C via cross-polarization (CP) since this is the most time efficient manner to move polarization from the electrons to the ¹³C. In principle we could have polarized ¹³C directly but the method is slower since spin diffusion in the ¹³C reservoir is slow. It will also be appreciated that the ¹³C signals could be indirectly detected via observation of 'H as is customary in many solution NMR experiments³⁸.

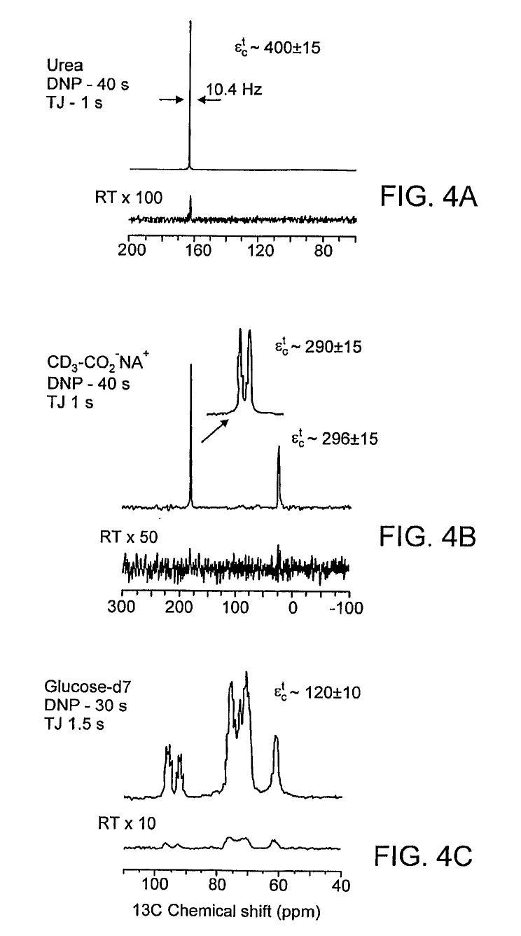

FIG. 4 shows the TJ-DNP enhanced ¹³C NMR spectra of (a) ¹³C-urea, Na[1,2-¹³C₂, ²H₃]-acetate and [U-¹³C,²H₇]-glucose in the top row of traces of the figures and the lower traces show the signal intensity obtained with ¹H decoupled Bloch decays for comparison. The enhancements observed in the spectra, which we label as ε^{†}, a definition that is discussed below, are included for each compound in the figure and are 400 for urea, 290 for sodium acetate and ˜120 for glucose. Note the ¹³C-¹³C J-coupling that is resolved in the acetate spectrum. This clearly establishes that, when the ¹³C T₁ is long compared to the melting period and is long in the solution phase, then it is possible to observe significant signal enhancements in the ¹³C solution spectra and that the resolution is not degraded by the presence of a paramagnetic polarizing agent.

We noted above that we have labeled the enhancements as ε^{†}, rather than ε as is common in solid state MAS experiments²⁻⁹. Thus, there are two conventions in use to report the size of enhancements that deserve explanation.

(1) In solid state magic angle spinning (MAS) experiments it is usual to compare the signal intensity in the presence and absence of microwave irradiation at the temperature where the DNP enhancement is performed. This ratio of signal intensities yields the enhancement ε due to the microwave irradiation. The data and enhancements reported in several other publications from our laboratory at T≧90 K use this convention and are due to the microwave driven enhancement alone ¹⁻⁹.

(2) In the case of liquids, however, the relevant enhancement, that we define as ε^{†}, is determined by the intensity of the DNP enhanced signal relative to the signal due to the Boltzmann polarization recorded at 300 K. Since the polarization is generated at low temperature, for example 90 K, there is an additional factor of (T_{obs}/T_{μwave})˜(300 K/100 K)=3 included in the calculation of the enhancement ε^{†}. When the polarization is performed at 1.2 K and the observation is at 300 K, this number increases to 250. Thus, enhancements reported in the literature for solid state and liquids experiments differ by the factor (T_{obs}/T_{μwave}), which can be substantial. For example, by polarizing at 1.2 K Ardenkjaer-Larsen and coworkers 3 reported ε^{†}=44,400 which corresponds to ε=178 if we take (T_{obs}/T_{μwave})=250. Accordingly, in this specification we quote two enhancements ε^{†} and ε that are related by ε^{†}=ε (T_{obs}/T_{μwave}) where T_{obs} and T_{μwave} are the temperatures where the signal observation and microwave irradiation occur. Note that, ε^{†}=ε in the limit where T_{obs}=T_{μwave.}

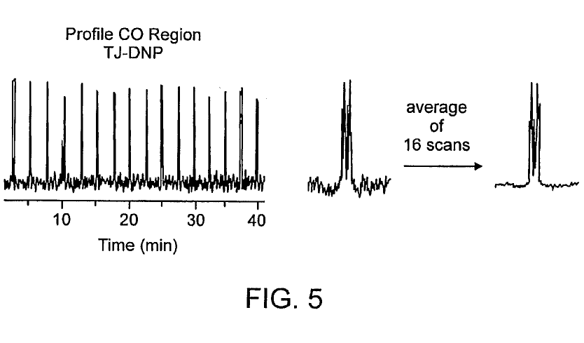

In addition, there are several features of the experiments described here that differ in significant ways from the experiments described by Ardenkjaer-Larsen, et al³⁹. In particular, while we performed an in situ TJ melting experiment, they in contrast utilized an approach involving polarization at low field, “dissolution” of the sample, and transfer to a higher field for observation. The difference in the experimental details is as follows. First, in the dissolution experiment the polarization step was performed at 1.2 K rather than 90 K. Second, it was performed in a 3.35 T field using a 200 mW, 94 GHz microwave source to drive the DNP method. Third, the triphenylmethyl based trityl radical⁴⁰ was the polarizing agent, and the ¹³C spins in the sample were polarized directly (ε˜178) rather than through the ¹H's. Because of the low temperature, the low microwave power, the long T₁ₑ of the trityl radical and the fact that they polarized ¹³C directly, their polarization times were ˜80 minutes. In contrast, we are able to achieve enhancements ε ˜290 in 40 s at 90 K at 5 T using our 140 GHz microwave source and biradical polarizing agents. Finally, in the “dissolution” experiment the sample, consisting of 40-50 mg of frozen polarized pellets, is melted and dissolved in 7 ml of hot water, diluting it by a factor of 150. If the polarized solute is used in imaging experiments, then dilution of the sample with solvent may not be a concern. However, for analytical NMR experiments it is clearly undesirable. Following dissolution, the sample was manually shuttled to a 400 MHz liquids spectrometer where solution NMR spectrum was recorded. Because of the requirement of shuttling to a second magnet, it is not possible to rapidly repolarize the sample. In the results illustrated in FIG. 4, the melting and spectroscopy are performed in situ. Further, the sample is not diluted since the melting is performed with a 10.6 μm laser light. Finally, since the polarization and observation is performed in situ, it can be refrozen, repolarized, etc. and the experiment recycled in the manner that is customary in analytical NMR experiments. The point is illustrated in FIG. 5, where we show a series of 16 spectra acquired over a period of ˜40 minutes from a sample of ¹³C-proline that was cycled through the steps: [polarization (40 s)—melting (1 s)—acquisition (100 ms)—refreezing (90 s)]ₙ. This result illustrates that the apparatus is sufficiently stable to reproduce the intensities in the spectra to 5%.

We also mentioned above that in the spectra of the samples that normally contain protons, the ¹H's were substituted with ²H. The reason for this is that in the liquid phase, the glassy glycerol mixtures used to polarize the samples are very viscous. Consequently, the ¹H relaxation times are very short (milliseconds)⁴¹ and the short ¹H T₁ leads to relaxation of the ¹³C and loss of the ¹³C signal. However, preliminary experiments with solvent systems that exhibit lower viscosity in the liquid phase, and still form low temperature glasses that disperse the biradical, suggest that it should be possible to employ protonated molecules in the TJ-DNP experiments.

The results shown in FIG. 4 clearly indicate that, in its present form, TJ-DNP is capable of providing substantial enhancements in sensitivity in ¹³C and other spectra of small molecules. Thus, when the quantity of sample is small and it can be repeatedly frozen, polarized and melted, then TJ-DNP experiments could provide a means to acquire ¹³C spectra (or spectra of any other low-γ spin) with excellent signal-to-noise in relatively short periods of time. An area where the current experimental protocol may find wide application is in metabolic screening, a subject that is of intense interest in the pharmaceutical industry.

Our purpose here was to demonstrate the feasibility of using TJ-DNP for observing spectra of liquids with enhanced sensitivity. It will be appreciated that a number of variations on the exemplified methods can be envisaged. For example, one could readily perform the polarization at lower temperatures, improve the efficiency of the melting method, and/or perform the experiments in glassy solvents that have a lower viscosity at room temperature. For example, the spectra displayed in FIG. 4 were the result of polarizing at ˜90 K and even greater enhancements could be obtained by performing the experiments at 2 K. In addition, the TJ-DNP experiment could be integrated with single scan experiments⁴² to obtain high sensitivity multidimensional experiments in a fraction of a second.

We have demonstrated the possibility of observing sensitivity enhanced ¹³C spectra of small molecules by first polarizing the sample and then melting it with laser radiation followed by observation of the solution NMR spectrum. Currently, we utilize biradical polarizing agents and gyrotron microwave sources for the DNP method. The latter enables the experiment to be performed in situ and to be recycled for signal averaging as is customary in conventional time domain NMR spectroscopy. The sensitivity enhancements at room temperature where the spectra are observed are presently 250, but they could be further enhanced by performing the polarization step at even lower temperature.

### Equivalents

The section headings used herein are for organizational purposes only and are not to be construed as limiting the subject matter described in any way.

While the present invention has been described in conjunction with various embodiments and examples, it is not intended that the present invention be limited to such embodiments or examples. On the contrary, the present invention encompasses various alternatives, modifications, and equivalents, as will be appreciated by those of skill in the art.

While the present invention has been particularly shown and described with reference to specific illustrative embodiments, it should be understood that various changes in form and detail may be made without departing from the spirit and scope of the present invention. Therefore, all embodiments that come within the scope and spirit of the present invention, and equivalents thereto, are intended to be claimed.

### REFERENCES

### (1) Hall, D. A.; Maus, D. C.; Gerfen, G. J.; Inati, S. J.; Becerra, L. R.; Dahlquist, F. W.;

Griffin, R. G. Science 1997, 276,930-932.

(2) Hu, K.-N.; Yu, H.-h.; Swager, T. M.; Griffin, R. G. J. Am. Chem. Soc. 2004, 126, 10844-10845.
(3) Gerfen, G. J.; Becerra, L. R.; Hall, D. A.; Griffin, R. G.: Temkin, R. J.; Singel, D. J. J. Chem. Phys. 1995, 102, 9494-9497.
(4) Becerra, L. R.; Gerfen, G. J.; Temkin, R. J.; Singel, D. J.; Griffin, R. G. Phys. Rev. Lett. 1993, 71, 3561-3564.
(5) Bajaj, V. S.; Farrar, C. T.; Mastovsky, I.; Vieregg, J.; Bryant, J.; Elena, B.; Kreischer, K. E.; Temkin, R. J.; Griffin R. G. J. Magn. Reson. 2003, 160, 85-90.
(6) Farrar, C. T.; Hall, D. A.; Gerfen, G. J.; Rosay, M.; Ardenkjaer-Larsen, J. H.; Griffin, R. G. J. Magn. Reson. 2000, 144, 134-141.
(7) Rosay, M.; Zeri, A. C.; Astrof, N. S.; Opella, S. J.; Herzfeld, J.; Griffin, R. G., J. Am. Chem. Soc. 2001, 123, 1010-1011.
(8) Rosay, M.; Lansing, J. C.; Haddad, K. C.; Bachovchin, W. W.; Herzfeld, J.; Temkin, R. J.; Griffin, R. G. J Am. Chem. Soc. 2003, 125, 13626-13627.
(9) Van Der Wel, P.; Hu, K.-N.; Lewandowski, J.; Griffin, R. G. in preparation.
(10) Duckett, S. B.; Sleigh, C. J. Prog. Nucl. Magn. Reson. Spectrosc. 1999, 34, 71-92
(11) Natterer, J.; Bargon, J. Prog. Nucl. Magn. Reson. Spectrosc. 1997, 31, 293-315.
(12) Navon, G.; Song. Y. Q.; Room, T.; Appelt, S.; Taylor, R. E.; Pines, A. Science 1996, 271, 1848-1851.
(13) Fitzgerald, R. J.; Sauer, K. L.; Happer, W. Chem. Phys. Lett. 1998, 284, 87-92.
(14) Cherubini, A.; Payne, G. S.; Leach, M. O.; Bifone, A. Chem. Phys. Lett. 2003, 371, 640-644.
(15) Pavloskaya, G. E.; Cleveland, Z. I.; Stupic, K. F.; Basaraba, R. J.; Meersmann, T. Proc. Natl. Acad. Sci. U.S.A. 2005, 102, 18275-18279.

### (16) Goto, A.; Hashi, K.; Shimizu, T.; Miyabe, R.; Wen, X. G.; Ohki, S.; Machida, S.; Tijima, T.; Kido, G. Physical Review B 2004, 69.

(17) Barrett, S. E.; Tycko, R.; Pfeiffer, L. N.; West, K. W. Phys. Rev. Lett. 1994, 72, 1368-1371.
(18) Michal, C. A.; Tycko, R. Phys. Rev. Lett. 1998, 81, 3988-3991.
(19) Zysmilich, M. G.; McDermott, A. J Am. Chem. Soc. 1996, 118, 5867-5873.
(20) Polenova, T.; McDermott, A. E. J. Phys. Chem. B 1999, 103, 535-548
(21) Prakash, S.; Alia; Gast, P.; deGroot, H. J. M.; Jeschke, G.; Matysik, J J. Am. Chem. Soc. 2005, 127, 14290-14298.
(22) Goez, M.; Mok, K. H.; Hore, P. J. J. Magn. Reson. 2005, 177, 236-246.

### (23) Afeworki, M.; McKay, R. A.; Schaefer, J. Macromolecules 1992, 25, 4084-4091.

### (24) Afeworki, M.; Schaefer, J. Macromolecules 1992, 25, 4092-4096.

### (25) Afeworki, M.; Schaefer, J. Macromolecules 1992, 25, 4097-4099.

### (26) Afeworki, M.; Vega, S.; Schaefer, J. Macromolecules 1992, 25, 4100-4106.

(27) Singel, D. J.; Seidel, H.; Kendrick, R. D.; Yannoni, C. S. J. Magn. Reson. 1989, 81, 145-161.
(28) Wind, R. A.; Duijvestijn, M. J.; Vanderlugt, C.; Manenschijn, A.; Vriend, J. Prog. Nucl. Magn. Reson. Spectrosc. 1985, 17, 33-67.
(29) Weis, V.; Bennati, M.; Rosay, M.; Griffin, R. G. J. Chem. Phys. 2000, 113, 6795-6802.
(30) Atsarkin, V. A. Sov. Phys. Usp. 1978, 21, 725-744,
(31) Overhauser, A. W. Phys. Rev. 1953, 92-411.
(32) Carver, T. R.; Slichter, C. P. Phys. Rev. 1953, 92-212.
(33) Loening, N. M.; Rosay, M.; Weis, V.; Griffin, R. G. J. Am. Chem. Soc. 2002, 124, 8808-8809.
(34) Hu, K.-N.; Song, C.; Swager, T. M.; Griffin, R. G. in preparation.

### (35) Joye, C. D.; Griffin, R. G.; Hornstein, M. K.; Hu, K.-N.; Kreischer, K. E.; Rosay, M.; Shapiro, M. A.; Sirigiri, J. R.; Temkin, R. J.; Woskov, P. P. IEEE Special Issue on High Power Microwave Generation 2006, In Press.

(36) Ferguson, D. B.; Krawietz, T. R.; Haw, J. F. J. Magn. Reson. Ser. A 1994, 109, 273-275.
(37) Ferguson, D. B.; Haw, J. F. Anal. Chem. 1995, 67, 3342-3348.
(38) Bodenhausen, G.; Ruben, D. J. Chem. Phys. Lett. 1980, 69, 185-189.

### (39) Ardenkjaer-Larsen, J. H.; Fridlund, B.; Gram, A.; Hansson, G.; Hansson, L.; Lerche, M.

H.; Servin, R.; Thaning, M.; Golman, K. Proc. Natl. Acad. Sci. U.S.A. 2003, 100, 10158-10163.

(40) Reddy, T. J.; Iwama, T.; Halpem, H. J.; Rawal, V. H. J. Org. Chem. 2002, 67, 4635-4639.
(41) Bloembergen, N.; Purcell, E. M.; Pound, R. V. Phys. Rev. 1948, 73, 679-712.
(42) Frydman, L.; Scherf, T.; Lupulescu, A. Proc. Natl. Acad. Sci. U.S.A. 2002, 99, 15858-15962.

What is claimed is:

1. A method comprising steps of:
providing a frozen sample in a magnetic field, wherein the frozen sample includes a polarizing agent with at least one unpaired electron and an analyte with at least one spin half nucleus;
polarizing the at least one spin half nucleus of the analyte by irradiating the frozen sample with radiation having a frequency that excites electron spin transitions in the at least one unpaired electron of the polarizing agent;
melting the frozen sample to produce a molten sample; and
detecting nuclear spin transitions in the at least one spin half nucleus of the analyte in the molten sample.

2. The method of claim 1, further comprising a step of:
freezing a sample in a magnetic field to provide the frozen sample in the magnetic field.

3. The method of claim 2, wherein in the step of freezing, the sample is cooled to a temperature of less than about 200 K.

4. The method of claim 2, wherein in the step of freezing, the sample is cooled to a temperature in the range of about 1 K to about 100 K.

5. The method of claim 2, wherein in the step of freezing, the sample is cooled to a temperature of about 90 K.

6. The method of claim 2, wherein the step of freezing is completed in less than about 2 minutes.

7. The method of claim 2, wherein the step of freezing is completed in less than about 1 minute.

8. The method of claim 2, further comprising repeating the freezing, polarizing, melting and detecting steps at least once.

9. The method of claim 1, wherein the at least one spin half nucleus is a ¹H nucleus.

10. The method of claim 1, wherein the at least one spin half nucleus has a γ-value smaller than that of ¹H and the step of polarizing further comprises irradiating the frozen sample with radiation having a frequency that causes cross-polarization between a ¹H nucleus present in the sample and the at least one spin half nucleus of the analyte.

11. The method of claim 10, wherein the at least one spin half nucleus is a ¹³C nucleus.

12. The method of claim 10, wherein the at least one spin half nucleus is a ¹⁵N nucleus.

13. The method of claim 10, wherein the ¹H nucleus present in the sample is from ¹H₂O

14. The method of claim 1, wherein the magnetic field has a strength in the range of about 0.1 T to about 30 T.

15. The method of claim 1, wherein the magnetic field has a strength of about 5 T.

16. The method of claim 14, wherein the radiation has a frequency in the range of about 2.8 GHz to about 840 GHz.

17. The method of claim 15, wherein the radiation has a frequency of about 140 GHz.

18. The method of claim 1, wherein in the melting step, the frozen sample is exposed to radiation having a wavelength of less than about 100 μm.

19. The method of claim 1, wherein in the melting step, the frozen sample is exposed to radiation having a wavelength in the range of about 0.5 μm and about 50 μm.

20. The method of claim 18, wherein the radiation is from a laser.

21. The method of claim 20, wherein the laser is a CO₂ laser.

22. The method of claim 18, wherein the radiation is from a lamp.

23. The method of claim 18, wherein in the melting step, the frozen sample is exposed to the radiation using an optical fiber.

24. The method of claim 18, wherein in the melting step, the frozen sample is within a cylindrical rotor.

25. The method of claim 24, wherein the cylindrical rotor is made of quartz.

26. The method of claim 24, wherein the cylindrical rotor is spun during at least the step of melting.

27. The method of claim 1, wherein the step of melting is completed in less than about 1 second.

28. The method of claim 10, wherein in the step of detecting, the at least one spin half nucleus is decoupled from ¹H nuclei present in the sample.

29. The method of claim 1 wherein the polarizing agent is a monoradical.

30. The method of claim 29, wherein the polarizing agent is a nitrogen oxide radical.

31. The method of claim 29, wherein the polarizing agent is a trityl radical.

32. The method of claim 1, wherein the polarizing agent is a biradical.

33. The method of claim 32, wherein the polarizing agent is bis-TEMPO-2-ethyleneglycol.

34. The method of claim 32, wherein the polarizing agent is 1-(TEMPO-4-oxy)-3-(TEMPO-4-amino)-propan-2-ol.

35. The method of claim 1, wherein the analyte is a protein or nucleic acid.

36. The method of claim 1, wherein the analyte is a metabolite.

37. The method of claim 36, wherein the metabolite is present in the sample at a concentration of less than 1 μM.

38. The method of claim 1, wherein the analyte is an imaging agent with a spin half nucleus that has a T₁ relaxation time of at least 6 seconds at 310 K in D₂O in a magnetic field of 7 T.

39. The method of claim 38 further comprising a step of administering at least a portion of the molten sample that includes the analyte to a subject before the step of detecting.

40. The method of claim 39, wherein in the step of detecting, a spatial distribution of the analyte within the subject is imaged by MRI.
